(19) United States
(12) Patent Application Publication
(10) Pub. No.: US 2026/0131080 A1
(43) Pub. Date: May 14, 2026

(54) LABELING ARRANGEMENT FOR A MULTI-PART CONTAINER, SYSTEM AND METHOD FOR ATTACHING A LABELING ARRANGEMENT TO A MULTI-PART CONTAINER

(71) Applicant: Schreiner Group GmbH & Co. KG, Oberschleissheim (DE)

(72) Inventors: Christian LIEBL, Westbury, NY (US); Andreas HOFENAUER, Germering (DE); Arne REHM, Rohrbach (DE)

(73) Assignee: Schreiner Group GmbH & Co. KG, Oberschleissheim (DE)

(21) Appl. No.: 19/118,415

(22) PCT Filed: Oct. 13, 2023
(86) PCT No.: PCT/EP2023/078478

§ 371 Date: Apr. 4, 2025

Related U.S. Application Data
(60) Provisional application No. 63/416,120, filed on Oct. 14, 2022.

(51) Int. Cl. A61M 5/31; B65C 3/06; B65D 51/24; B65D 55/08

(52) U.S. Cl. CPC A61M 5/31; B65C 3/06; B65D 51/245; B65D 55/0818; A61M 2005/3104; A61M 2205/60

(57) ABSTRACT

A labeling arrangement includes a sleeve-shaped cap element having a wall with an inner surface which establishes a first contact surface and which is adapted to be coupled to an outer surface of a closure element of the container by an interference fit relative to a longitudinal axis of the container and to surround the closure element.

The present invention relates to a labeling arrangement that provides reliable labeling for a container in a simple and cost-effective manner. The invention also relates to a system comprising such a labeling arrangement and a method for attaching such an electronic labeling arrangement to a multi-part container.

Labelling arrangements usually comprise a label which can be used for authorization and authentication or for proof of origin. In particular, such labelling arrangements are used to provide information on contents or for traceability, or when it is necessary to protect ingredients against unauthorized access. This applies, among other things, to containers in the pharmaceutical and medical sector, the contents of which need to be labeled and protected.

It is an objective underlying the invention to contribute to reliable and convenient labeling of a container in a simple and cost-effective manner.

The objective is achieved by the respective features of the independent patent claims. Advantageous embodiments are given in the respective dependent patent claims.

According to one aspect of the invention, a labeling arrangement for a multi-part container has a sleeve-shaped cap element comprising a wall with an inner surface which establishes a first contact surface. The first contact surface is designed to be coupled to an outer surface of a closure element of the container with respect to a longitudinal axis of the container by means of an interference fit and to surround the closure element.

The labeling arrangement described can be used to reliably label syringes, among other things. As an adapter sleeve, the cap element provides a labeling surface so that a label can be applied reliably and stably in an upper and a lower area of the container. The cap element can, for example, surround a narrow ring-shaped projection on an upper side or end face of the closure element, which is present in commercially available Luer-Lock closures. The cap element is essentially only applied to an upper area of the closure element, which is usually narrower than the structures located underneath, and is designed in particular in such a way that it does not protrude outwards or protrudes particularly little compared to the widest structures.

In the context of this description, terms such as “top” and “bottom” as well as “inside” and “outside” refer to an operational arrangement or application of the labeling arrangement and the container. This also applies to terms such as “head” and “body”, whereby the head is usually located at the top and the body is usually located at the bottom. The closure element forms a primary closure of the container, for example an upper part of an injection vial or a syringe, and comprises a head part and a threaded part, for example based on a Luer-Lock syringe, which can be screwed together. The head part is unscrewed and removed from the threaded part to open the container. The container body forms a lower part of the injection vial or syringe and realizes a container for holding and storing a predetermined content.

The cap element realizes an adapter cap, especially for narrow containers, such as thin syringes, and is placed or pressed onto the closure element from above. The dimensions of the cap element are in particular adapted to the intended use on the closure element of the container, so that the first contact surface can be reliably pressed on around the outside of the closure element. In this context, containers or syringes are referred to as thin or narrow where there is approximate circumferential equality between the closure element and the body of the container. This is usually the case with pharmaceutical or medical containers, which have a relatively small diameter and are correspondingly relatively narrow or thin. In other words, the labeling arrangement described is particularly suitable for containers whose circumferential geometries are very similar at the top and bottom and only differ from each other by up to two millimeters in diameter, for example. However, the labeling arrangement can also be useful for differently shaped containers. Even small deviations in circumference can lead to a disadvantageous labeling capability and cause mislabeling, so that, for example, fully automatic operation for applying the label is not possible.

Using the described labeling arrangement, a useful circumferential compensation can be formed even with narrow containers, such as a thin syringe with Luer-Lock adapter or integrated Luer-Lock, thus contributing to improved labelability. For example, the container is designed as a plastic or COC syringe in which the Luer-Lock closure is also formed during injection molding. Alternatively, the container is realized as a glass syringe, for example, in which a Luer adapter is clipped or pressed on. In relation to an applied state where the labeling arrangement is attached to the container, a label can be reliably attached and securely bonded both at the top in the area of the container head and at the bottom in the area of the container body. The label can be sealed at the top and bottom without or at least with minimal wrinkling using the labeling arrangement described. An intermediate area, for example with a syringe shoulder and/or a threaded element, can thus be bridged, closed or sealed, in particular to counteract unwanted penetration of dirt, gas, vapor and/or light.

The labelling arrangement can improve the RFID functionality of syringes or enable RFID functionalization at all, especially of thin syringes. The additional labeling surface created by the cap element makes it possible to arrange the RFID transponder inlay or the antenna structure further up on the syringe and thus to move it partially or completely out of the area of the syringe body that is operationally filled with a liquid. This can have a particularly beneficial effect on the performance of the RFID functionality. Thus, reliable and convenient electronic labeling of a container can be realized in a simple and cost-effective way even on a very limited area of the container, for example a Luer-Lock syringe with a relatively small radius. In addition, the described labeling arrangement is also suitable for other primary packaging or containers in the pharmaceutical and/or medical field, such as injection vials or vials.

According to a preferred embodiment of the labeling arrangement, the cap element has an inner projection that establishes a second contact surface. The first and second contact surfaces are formed in coordination with each other and in coordination with an annular projection on an upper side or end face of the closure element and provide a coupling structure in the form of a crimp groove or press groove, which can be reliably coupled to the annular projection by means of a press fit. The first and/or the second contact surface are preferably annularly closed in relation to the longitudinal axis, so that the press groove establishes a circumferential channel or trench into which the raised projection of the closure element can be pressed. The press groove is preferably slightly narrower than the width of the projection on the end face, so that a reliable press fit of the cap element on the closure element can be established.

Furthermore, the first and/or the second contact surface can be designed with a predetermined inclination in relation to the longitudinal axis and taper towards the other contact surface in the direction of one end face of the cap element. For example, both contact surfaces are tapered so that the press groove tapers upwards on both sides. Alternatively, one or both contact surfaces are aligned parallel to the longitudinal axis and/or to each other. Furthermore, one or both contact surfaces can alternatively be designed to taper downwards, so that the press groove tapers downwards on one or both sides.

The cap element can be closed on its end face or, according to an embodiment, have a penetrating opening, for example in the form of a central recess on the end face. Such an opening can provide a channel for fluid exchange between an inner space and an outer space of the cap element. This allows gas or liquid to enter and exit, which is useful, for example, during a sterilization process between an inner space and an outer space of the cap element. This is advantageous, for example, for prefilled syringes that are sterilized with hydrogen peroxide, water vapor or ethylene oxide (ETO) gas, so that the sterilization medium can pass through the recess of the cap element to the primary closure. Furthermore, such a fluid opening may, for example, be provided in a side wall of the cap element and enable an exchange of media. In addition, a penetrating opening can ventilate an intermediate space between the closure element and the attached cap element and help to ensure that undesired media entrapment in the intermediate space has no adverse effects on the container and its contents. In addition, a possibility for pressure equalization of an otherwise possibly hermetically sealed system can thus be established.

According to a further embodiment of the labeling arrangement, the cap element has a beveled guide surface on an underside of the wall, for example adjacent to the first contact surface, for placement on the closure element. Accordingly, the underside of the wall can have a chamfer that is inclined upwards towards the longitudinal axis and enables the cap element to be placed and guided more easily. Furthermore, such a guide surface can be designed to match an outer contour of the closure element in order to establish a flush transition between the cap element and the closure element. Commercially available Luer-Lock closures in particular have handle bars that are rounded towards the top and become narrower. The guide surface of the cap element can be adapted accordingly and placed on the rounded, narrower area of the handle bars and contribute to increased circumferential uniformity. Furthermore, an end position of the cap element on the closure element can thus also be indicated.

If the cap element has an inner protrusion which, for example, fills the space within the annular protrusion of the closure element, the inner protrusion can also have a rounded portion on an underside, for example adjacent to the second contact surface, which can contribute to simplified placement on the closure element.

According to a further embodiment of the labeling arrangement, the cap element has a first contact surface and possibly also a second contact surface on both an upper side and a lower side of the wall, which are separated from each other by an end wall. Accordingly, the cap element can be coupled to the outer surface of the closure element by means of a press fit, both on the top side and on the bottom side in relation to the longitudinal axis of the container. In this way, the cap element can be used on both sides and enables a simplified application process, as it is no longer necessary to differentiate between the top and bottom when aligning the cap element. The cap element can be designed inversely and thus have the same geometry of the first and/or second contact surface at the top and bottom. Alternatively, the cap element can have a different geometry at the top than at the bottom and, for example, have contact surfaces that converge at the top, while the contact surfaces at the bottom are oriented parallel to each other or only the first contact surface is present at the bottom. Accordingly, the end wall forms a central partition wall between the two attachment options of the cap element and, when pressed onto the closure element, nevertheless forms an end wall of such a system.

According to a further embodiment of the labeling arrangement, the cap element has a web structure which is designed to match an outer contour of the closure element of the container and comprises a plurality of webs. The webs are designed to engage in corresponding spaces between the handle webs of the closure element. Such a cap element is usually present in Luer-Lock syringes, so that a larger and standardized labelling surface can be formed on the Luer-Lock syringe by means of the specially designed cap element.

In addition, one or more bars of the bar structure can have a latching element in the form of a latching bar or a latching protrusion, for example in the form of a latching hook. The latching web connects two or more webs together on an underside, for example, and forms a predetermined protruding edge on an inner side. A latching projection can be formed at a free end of a web and also form a predetermined raised edge on the inside. The latching web and/or the latching projection are designed in such a way that they can engage in a groove of the closure element of the container, so that a retaining force can be set in the direction of the longitudinal axis in relation to the longitudinal axis between the cap element and the closure element.

Alternatively or additionally, the labeling arrangement can be further designed in such a way that the cap element has a circumferential contour in relation to the longitudinal axis, which forms a negative shape to a circumferential contour of the closure element. By means of such an inverse shaping of the inner contour of the cap element, a particularly reliable hold on the closure element of the container can be established. The cap element forms a form-fitting cap, which provides geometric compensation in relation to the closure element and can also be subsequently applied to existing syringes or other containers in order to provide them with improved labeling capability. In particular, a lower area of the cap element can be designed as a negative shape of a syringe cap or the closure element, so that the contact surface can be adjusted and, in addition to simpler application, a maximum flush surface can be formed.

The cap element is designed in particular in such a way that it only extends in the area of the closure element and only establishes a short adapter cap on top of the closure element. As the cap element is preferably intended for thin containers, it has a correspondingly thin wall. The wall of the cap element can have a thickness of 0.5 to 1.5 millimeters in relation to a radial expansion transverse to the longitudinal axis. Alternatively, the wall can also have a thickness of only 0.3 or 0.4 millimeters. The wall of the cap element compensates for the diameter of the Luer-Lock closure, onto which the cap element is pressed, in relation to the syringe body. In addition, the wall can also be designed with a greater wall thickness than 1.5 millimetres in order to compensate for circumferential differences. For example, the wall of the cap element can have a thickness of up to 3 millimeters.

According to a further embodiment, the labeling arrangement comprises a label, wherein the label and the cap element are designed in coordination with one another and are coupled to one another, so that the label can be attached to the closure element of the container by means of the cap element. In particular, the label is designed as an electronic label with an RFID functional unit that has an RFID chip and an antenna structure coupled to it.

The labeling arrangement can comprise an opening detection element that is arranged in a separation area of the label. For example, a perforation is provided so that, in relation to a state in which the labeling arrangement is attached to the container, an opening of the container can be indicated by removing at least one partial element of the label from the container. The removable partial element can be designed as a label section and/or a conductor path section of the RFID functional unit and indicate a visual and/or electronic opening. When the container is opened, the label is separated, for example, along a predetermined perforation and separated from the second sub-element by means of pulling and/or twisting and pulled off or twisted off upwards together with the primary closure or a part thereof and removed from the container body. A perforation acts as a severing element and can in particular also be combined with punch-outs or branches upwards and/or downwards, for example to leave clearly visible damage after severing the label and opening the container. The perforation can be serrated, for example, in order to clearly indicate initial opening. Alternatively or additionally, the label can have a film or film element in the separation or cut-through area that has a predetermined tear resistance.

The separation area of the label should preferably be attached to the container in such a way that it is assigned to an area that corresponds to a transition of the closure element and an adjacent element, for example a threaded element. The separation area can then also be referred to as the severing area, as it enables the continuous label to be severed in a targeted manner. Alternatively, the separation area is formed in coordination with the container in an area which, in an applied state of the labeling arrangement on the container, is assigned to a transition between a head part, for example a threaded element, and the adjacent container body. Thus, a sufficiently forceful turning and/or pulling of the additional element simultaneously leads to a predetermined separation of the labeling arrangement. However, the separation area can also be arranged in other areas.

By means of such a separation area or an opening detection element, tamper protection can be set up in addition to convenient labeling of the container, which prevents or at least clearly indicates opening of the container or tampering with the closure element. Tampering or an opening attempt then leads to a clearly visible tearing of the label and/or destruction of a conductor track of the RFID label. In the event of a severed conductor path, the RFID chip can thus detect an opening attempt in the form of a change in electrical conductivity and/or signal strength and provide an electronic opening detection element. Such an opening attempt can be stored in the RFID chip and read out as required. Alternatively or additionally, the RFID functional unit can be set up in such a way that it can either no longer be read or can only be read with a significantly reduced read range after the conductor track has been cut, which clearly indicates an initial opening.

Preferably, an adhesive-free surface is formed in the cut-through area of the label. This makes it easier to separate the label and remove the respective upper sections. The force required to separate the closure element with the cap element and the label is reduced compared to fully bonded components and the opening dynamics of the container applied with the labeling arrangement are improved.

The cap element is designed, for example, as a circumferentially closed sleeve with an end wall and can be pushed or pressed onto the closure element. Alternatively, the cap element can be slotted on one side wall and/or form an upwardly open ring sleeve. The pressed-on cap element is in direct contact with the outer surface of the closure element, at least in sections, so that a reliable coupling in the form of an interference fit is already established. The cap element is preferably rotationally symmetrical with respect to the longitudinal axis and can therefore be pressed onto the closure element regardless of rotation. If a web structure with a latching element is provided, the cap element is pressed on until the latching element engages and establishes a holding force in the direction of the longitudinal axis. As an alternative or in addition to the web structure and/or in addition to the press fit, the cap element can be connected to the closure element by means of gluing and/or welding. When the container is opened, only the holding force of the primary closure or the closure element on the container body needs to be overcome by turning and/or pulling. Furthermore, the cap element can be specifically extended upwards so that an even larger labeling surface can be formed on the container.

The cap element is preferably formed from a plastic by injection molding and/or 3D printing. The cap element is preferably made of plastic, such as polypropylene (PP), polyethylene terephthalate (PET), polycarbonate, cycloolefin polymers (COC) or cycloolefin copolymers (COP). The cap element can thus be produced cost-effectively and has a wall thickness of, for example, 0.5 millimetres or more on the circumferential side wall, which is sufficient for production and mechanical stability, without expanding the diameter of the closure element in the uppermost area beyond the diameter of the container body. As an adapter cap, the cap element thus creates a surface in the uppermost area of the closure element that is sufficient for a label to adhere and that is circumferentially identical or similar to the circumference of the container body. Preferably, an outer surface of the cap element is roughened and/or has a microstructure that contributes to a particularly reliable adhesion of the label.

For example, an outer surface of the cap element is designed for the adhesion of an RFID label equipped with a pressure-sensitive adhesive. This can be achieved by selecting a plastic with a high surface energy and/or roughness of the surface. The outer surface of the cap element can also have a corrugation, for example, which provides a gripping structure for performing an opening rotary movement. The label can also provide a certain roughness or grip and contribute to an improved feel of the container provided with the labeling arrangement. Useful surface properties can be provided.

The labeling arrangement preferably comprises an adhesive layer arranged on an underside. If an RFID functional unit is provided, this enables information to be read electronically. In conjunction with a functional carrier, such as a carrier film, the RFID functional unit can realize an RFID transponder or an RFID tag, which can form a so-called “soft tag” as a bendable component. In conjunction with a component that is not significantly bendable, an RFID tag can form a so-called “hard tag”. The label is therefore particularly preferably realized as an RFID label and has a carrier layer with an adhesive layer so that the RFID label can be easily and reliably attached to the primary closure and/or the additional element by means of adhesive.

The RFID label or the label can be single-layered or multi-layered. For example, it can be a wrap-around or wrap-around label that partially or completely encloses a circumference of the closure element and, for example, lies on itself. In terms of height or length, the label can partially or completely cover the closure element from bottom to top. Furthermore, the label can extend from a foot end of the container body to a head end of the closure element.

The label can also comprise a reusable adhesive layer by means of which the label can be attached to the cap element and detached again, at least in sections. The reusable adhesive layer is preferably based on a reusable adhesive layer, so that a label section can be adhered to the cap element, detached again and adhered again. Alternatively or additionally, a reusable coupling between the label and the cap element can be established by means of electrostatic adhesion. In this way, for example, a label flag can be formed, which can provide a particularly useful readability of the RFID functional unit. In particular, the antenna structure is then preferably arranged in the label section that can be folded in and out for improved electronic readability. Alternatively or additionally, the RFID functional unit is designed as a multi-layer RFID label, for example as a label booklet, and has at least one section that can be folded up and in again, so that a label flag can be formed that protrudes from the remaining RFID label.

According to a further aspect, the invention comprises a use of an embodiment of the described labeling arrangement for a container that can be divided into several parts with a container body and a closure element, which in particular form components of a relatively narrow Luer-Lock syringe.

According to a further aspect of the invention, a system comprises a container divisible into a plurality of parts with a closure element and a container body which are coupled to each other. The system further comprises an embodiment of the labeling arrangement described above, which is coupled to the closure element such that the cap element is connected to the closure element by means of an interference fit and the inner, first contact surface of the cap element contacts and surrounds an outer surface of the closure element. The container of the system is preferably realized as a Luer-Lock syringe. In particular, the labeling arrangement described also enables subsequent sealing or electronic labeling of prefilled syringes. The cap element provides a reliably labelable upper end of the system and can thus contribute to a simple, automated and process-reliable labeling capability, even for particularly thin containers. As the cap element is designed in such a way that it is pressed onto the narrow annular projection of the closure element and extends into any existing gaps, the diameter of the closure element is not or only very slightly increased.

In that the use and the system relate to or comprise an embodiment of the described labeling arrangement, the described properties and features of the labeling arrangement are also disclosed for the use and for the system and vice versa.

According to a further aspect of the invention, a method for attaching an embodiment of the labeling arrangement described above to a closure element for a multi-part container comprises providing the container with the closure element and the container body coupled to each other. The method further comprises providing the sleeve-shaped cap element and placing and pressing the cap element onto the closure element so that the first contact surface contacts an outer surface of the closure element and the cap element is attached to and surrounds the closure element by means of an interference fit.

By means of the described application or assembly method, it is possible to use the properties and features provided by the labeling arrangement for secure protection and reliable and convenient electronic labeling of the container. Accordingly, the method is also suitable for forming an embodiment of the system described above. In that the method relates to an application or assembly of an embodiment of the described labeling arrangement and its arrangement on the container, the described properties and features of the labeling arrangement are also disclosed for the method and vice versa. The coupling of the closure element to the container body may be performed before or after the coupling of the cap element to the closure element. The method may further comprise providing and attaching a label such that the label is attached to the cap element and to the container body.

It is a finding in connection with the present invention that a surface of closure structures and, in the case of glass syringes, also the surface of a Luer-Lock closure are unfavorably or not usefully designed for the application of labels by means of pressure-sensitive adhesives. On the one hand, surfaces are not sufficiently large and flat and, on the other hand, the materials used may have a surface microstructure and surface tension that is unsuitable for pressure-sensitive adhesives. There are various reasons why it is useful to label pharmaceutical syringes from the syringe body to the primary closure. For example, this enables the following:
gain in labeling area for labeling and functionalities
implementation of an analog seal and/or a digital seal with RFID functionality
constructive solution to increase the performance of RFID functionalities
syringe sealing as part of light protection and/or gas barrier

Since closure elements or primary closures of the syringes available on the market are generally unsuitable for the adhesion of a label, for example due to a too small flat surface or an external corrugated structure, and are also usually not circumferentially identical to the syringe or container body, the described labeling arrangement with the cap element enables a simple and reliable option to achieve a sufficiently large adhesive surface and circumferential uniformity for stable and long-lasting labeling. The cap element can be reliably attached to the closure element provided for this purpose by means of a targeted press fit and does not require an additional interlocking structure on the inside or on the first contact surface.

The labeling arrangement described takes particular account of the large number of containers and syringe types available on the market in which the diameter of a syringe cap or closure is not relevantly smaller than the diameter of the associated syringe body. For example, such thin containers are realized as syringes with a Luer lock closure and 1 ml filling volume, in which the closure element and the syringe body are almost identical in circumference. The cap element of the described labeling arrangement makes it possible to provide an adapter cap for such thin syringes in a simple and cost-effective manner, which surrounds the closure element without exceeding the diameter of the syringe body. Thus, embodiments of the labeling arrangement enable one or more of the following advantages, among others:
as an adapter cap, the cap element creates a sufficiently large adhesive surface, which is circumferentially coextensive with the syringe body, for stable adhesion of a label.
the cap element as a whole or at least in the adhesive surface intended for a label is preferably not larger in circumference than the syringe body. If the cap element has components below the adhesive surface, such as the web structure described, this is preferably also not bulky and uses existing free spaces on the closure element.
the cap element has a sufficiently firm mechanical coupling with the syringe cap for force transmission in the rotational and axial directions. In particular, the labeling arrangement uses structural features on the top of the syringe cap or the closure element to achieve a stable and reliable coupling. It is therefore not necessary to glue the cap element directly or to design it in such a way that it encloses the entire closure element for a secure hold.

By means of the described labeling arrangements, reliable labeling of pharmaceutical containers can be realized, which can be applied to such closure structures of prefillable syringes, preferably by means of pressure-sensitive adhesive, without having to change the shape and applicability of the syringe. The described embodiments of the labeling arrangement enable both prior and subsequent attachment to a primary closure of a syringe before or after it is or has been attached to the syringe body. Existing syringes can be retrofitted with RFID functionality in a simple and cost-effective way using the labeling arrangement and the cap element.

An area between the cap element and the syringe body that is smaller than or equal to the diameter of the syringe body is spanned. Where the circumference is the same as that of the closure element and/or the syringe barrel, the label is additionally bonded over part of the surface. The height or length of the cap element along the longitudinal axis can also be adapted to the labeling surface requirements and can be made extra long, for example.

Particularly in the version with a crimping groove or press groove, the cap element enables reliable mechanical interlocking with the top or distal side of the syringe cap or the closure element. The crimping groove is particularly adapted to commercially available closure elements, which are designed as hard plastic components, for example, and have an annular protrusion. This allows a press fit to be set up on both sides of the protrusion, which provides a secure and stable hold so that the cap element is removed together with the syringe cap when the syringe is opened. The crimping groove of the cap element and the raised edge on the top of the syringe cap, which couples with the crimping groove, are independent of the rotational position. Accordingly, it is not necessary to consider an orientation in relation to structural features of the closure element when attaching the cap element.

To simplify the application of the cap element to or on the closure element, an inner side of the cap element in a lower closing area can be designed in such a way that it is significantly wider or offers more internal free space than in the upper area, which is firmly coupled to the syringe cap. A circumferential chamfer can also be formed in the lower area, which forms a kind of bevel so that the cap element can be fitted with a greater tolerance. Such a specifically designed taper at the bottom of the wall automatically achieves the correct end position when the cap element is pressed on and, with the appropriate pressure, a very firm connection is achieved by wedging. The cap element therefore has a self-adjusting design.

The flanks of the crimping groove, which are realized by the opposing first and second contact surfaces, can be parallel or inclined in relation to the syringe axis or the longitudinal axis. For example, both flanks are wedge-shaped and taper towards each other. It is also possible for only one flank to run at an angle to the syringe axis and for the other flank to be aligned straight along the syringe axis. It is also conceivable that both flanks run parallel in the axial direction. A press fit is then preferably achieved by making the groove gap of the press groove slightly thinner or narrower than the width of the annular edge on the syringe cap.

Even if the cap element with the syringe closure can primarily be made with structural features on the top of the syringe cap that are independent of the rotational position, it can also be beneficial to realize further toothing options. In this case, it can be useful to optionally utilize the spaces between the handle bars on the syringe cap by means of the described bar structure. Locking hooks or locking bars can engage in a lower groove of the closure element and despite such additional interlocking with lateral structural features of the syringe cap, the diameter of the syringe body is not exceeded by the specially designed cap element. In addition, the available adhesive surface for a label can be increased by means of the guide bars of the bar structure, which preferably fill the spaces between the grip bars flush.

Individual bars of such a bar structure can be connected to each other by means of the latching bars or latching hooks. This can be used, for example, to stabilize the bars and hooks so that they do not break when they are put in place and due to the associated mechanical load. In order to overcome the higher circumference in the area of the handle bars compared to the groove of the syringe cap, the locking hooks or the locking bars are preferably not fully connected to each other. Provided gaps or openings in the bar structure allow the cap element to move outwards in the area of the grip bars of the closure element when it is fitted. Once the individual latching hooks, the connected latching hooks or the latching bars have overcome the area of the grip bars of the syringe cap during attachment, they latch into the groove of the syringe cap and provide additional interlocking.

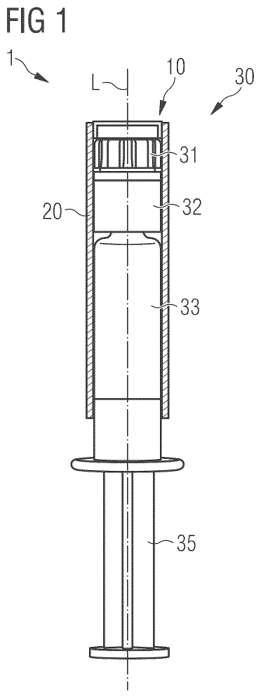

In the following, embodiments of the invention are explained with reference to schematic drawings. It shows:
FIG. 1 an embodiment of a system with a Luer-Lock syringe and a labeling arrangement attached thereto,

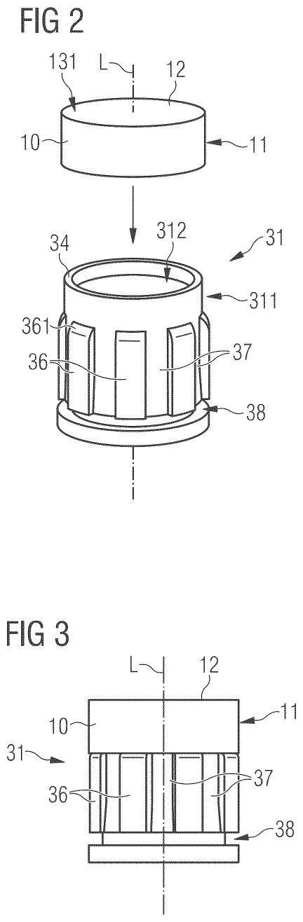

FIGS. 2-5 embodiments of a closure element and a cap element of the labeling arrangement provided therefor in various views,

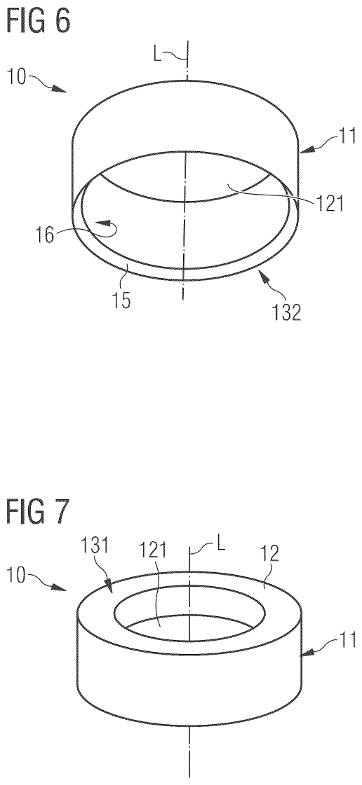

FIGS. 6-22 further embodiments of the cap element of the labeling arrangement in various views,

FIGS. 23-25 further embodiments of the system with a Luer-Lock syringe and a labeling arrangement attached thereto, and

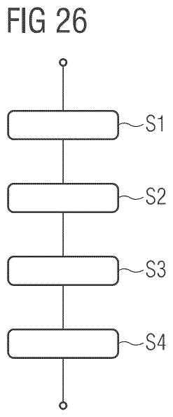

FIG. 26 a flowchart for a method of attaching the labeling arrangement to a multi-part container.

Elements of the same construction and function are marked with the same reference signs across the figures. For reasons of clarity, not all of the elements shown in all of the figures are marked with the corresponding reference symbols.

FIG. 1 shows a schematic side view of a system 1 with a multi-part container and a labeling arrangement attached to it. The container is designed as a Luer-Lock syringe 30 and comprises a primary closure with a closure element 31 and a Luer adapter or threaded element 32, which are coupled to a syringe body 33. For example, a content is arranged or can be arranged in the syringe body 33, which can be expelled from the syringe body 33 as required by means of a syringe plunger 35. In relation to a longitudinal axis L, the closure element 31 can also be referred to as the head part or syringe cap of the Luer-Lock syringe 30. The closure element 31 and the threaded element 32 realize a head-side end structure of the Luer-Lock syringe 30 as a primary closure. The labeling arrangement, which comprises a sleeve-shaped cap element 10 and a label 20, is arranged on the closure element 31. The cap element 10 and/or the label 20 may comprise an RFID functional unit with an RFID chip 21 and an antenna structure coupled thereto (see FIGS. 23-25).

As will be described below with reference to FIGS. 1-26, the labeling arrangement enables reliable and convenient labeling of a container, which can be realized in particular as a thin syringe, in a simple and cost-effective manner. The embodiments of the labeling arrangement described below refer to the Luer-Lock syringe 30 or more generally to a syringe 30, but which are also transferable to other pharmaceutical multi-part containers, such as injection vials or vials.

Terms such as “top” and “bottom” as well as “inside” and “outside” refer to an operational arrangement or application of the labeling arrangement according to the illustrated orientations of the syringe 30 according to FIG. 1. A head of a respective syringe is therefore arranged at the top and the body usually at the bottom. The closure element 31 forms a component of the respective syringe 30 as a head part, which is unscrewed and/or pulled off and removed from the syringe body 33 to open the syringe 30.

The cap element 10 is intended in particular to create a circumferential alignment with the syringe body 33 at one end of the syringe 30 on the head side, thus contributing to improved labeling capability. The cap element 10 takes into account an upper structure of the closure element 31, which is generally narrower than a structure located below it. The cap element 10 has a wall 13 with an inner surface which establishes a first contact surface 16 and which is designed to be coupled to an outer surface 311, 312 of the closure element 31 by means of an interference fit with respect to the longitudinal axis L of the syringe 30 and to surround the closure element 31 (see also FIGS. 9 and 11-17).

FIG. 2 illustrates in a perspective view how the cap element 10 is placed on the closure element 31 of the syringe 30. FIG. 3 shows in a further view the coupled state in which the cap element 10 is pressed onto the closure element 31. According to FIGS. 2 and 3, the cap element 10 has an outer surface or outer side 11 and an end wall or end face 12 and is designed as a sleeve that is open at the bottom and closed at the top. The outer side 11 realizes an outer circumferential surface and the first contact surface an inner circumferential surface of the wall 13 (see also FIG. 6). The front side 12 is formed on an upper side 131 of the wall 13, which is opposed by a lower side 132 of the wall 13 (see also FIGS. 6 and 7).

The cap element 10 is pressed with its wall 13 around an annular projection 34 of the closure element 31 and thus creates a circumferential uniformity or at least a circumferential similarity to the syringe body 33, so that the label 20 can be reliably attached to the top and bottom of the syringe 30. By means of the cap element 10, the label 20 can seal the syringe 30 at the top and bottom and bridge a transition area with the threaded element 32 as well as existing grip structures on the closure element 31. This reliably prevents dirt or other undesirable substances from entering the area of the closure element 31 or the threaded element 32.

The closure element 31 of the Luer lock syringe 30 usually comprises a handle structure with vertical handle bars 36 and intermediate spaces 37 as well as a groove 38 running horizontally around the bottom (see FIG. 2). The handle bars 36 taper upwards in the direction of the projection 34 and have a rounded portion 361. The cap element 10 is designed such that it utilizes at least the narrower projection 34 or, if necessary, also takes into account one or more of the structural features of the closure element 31 described above. A structure or surface finish on an outer side 311 or an end face 312 of the closure element 31 can also be included.

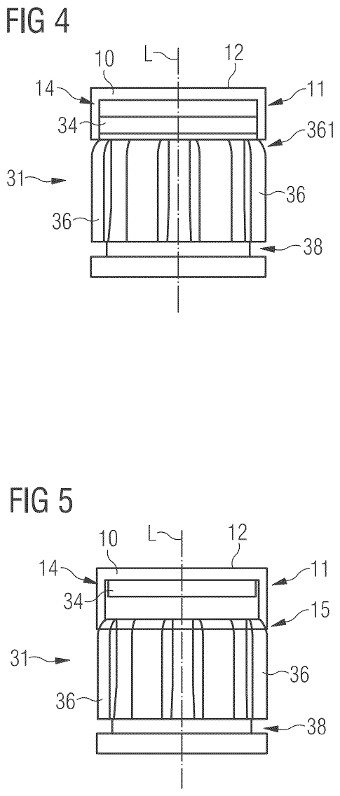

FIG. 4 shows, for example, the embodiment of the cap element 10 and the closure element 31 according to FIG. 3 in a transparent embodiment. In contrast, the cap element 10 in FIG. 5 has a specially designed guide surface and/or support surface 15 on the underside 132 of the wall 13 (see also FIGS. 6, 8 and 10). The guide and/or support surface 15 enables simplified positioning of the cap element 10 and, in addition, the rounded edges 361 of the handle bars 36 of the closure element 31 are taken into account and a transition between the cap element 10 and the handle bars 36 is designed to be as flush or contour-continuous as possible.

FIG. 6 illustrates in a schematic view from below a further embodiment example of the cap element 10 with a central penetrating opening 121 through the end face 12. Accordingly, the cap element 10 can be realized as a closed or open adapter cap, as in FIGS. 2, 8 and 10. FIG. 7 shows a schematic view from above of a further embodiment example of the cap element 10 with a central penetrating opening 121 through the end face 12, which is smaller than that in FIG. 6. The cap element 10 according to FIG. 7 can also be described as partially open. An opening in or through the cap element 10 may allow fluid exchange between an interior space and an exterior space of the cap element 10, which is beneficial, for example, during a sterilization process. Hydrogen peroxide, water vapor or ethylene oxide (ETO) gas can pass through the opening 121 of the cap element 10 as a sterilizing medium to the closure element 31.

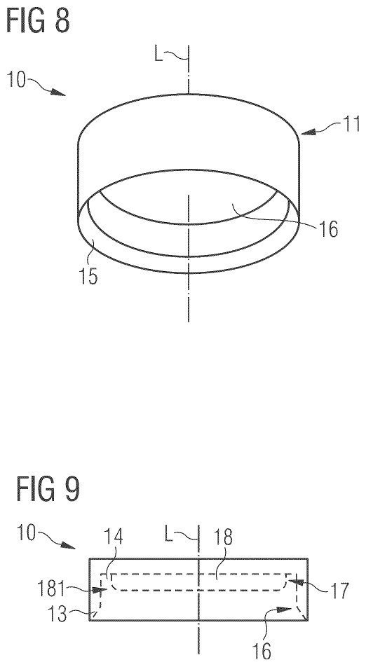

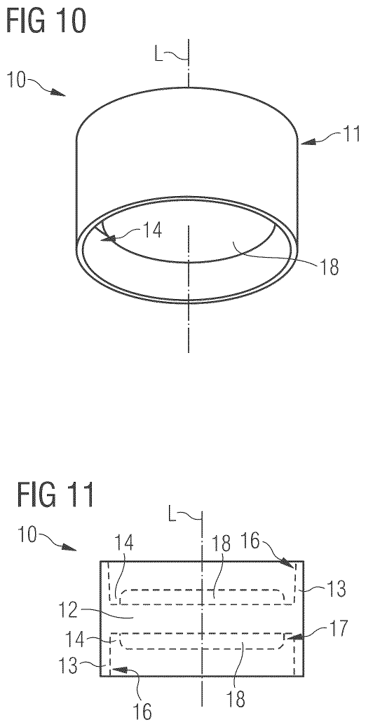

FIG. 8 illustrates a schematic view from below of an embodiment example of the cap element 10, which can be placed on one side of the closure element 31. In comparison, FIG. 10 shows an embodiment of the cap element 10 extended along the longitudinal axis L, for example to provide an enlarged adhesive surface for attaching the label 20. Alternatively, FIG. 10 can also illustrate a cap element 10 that can be attached on both sides, as described below with reference to FIG. 11.

FIG. 9 shows a schematic side view of a further embodiment of the cap element 10. The cap element 10 has an inner projection 18 which establishes a second contact surface 17. The second contact surface 17 is formed opposite the first contact surface 16 and delimits the inner projection 18 on a path with a smaller diameter than the circumferential first contact surface 16. The two contact surfaces 16 and 17 are formed in coordination with each other and establish a crimping groove or press groove 14, which is formed in coordination with the annular projection 34 on the end face 312 of the closure element 31. The contact surfaces 16, 17 enable the cap element 10 to be fixed on both sides to the projection 34 on the closure element 31. A width or a diameter of the inner projection 18 radial to the longitudinal axis L is thus matched to a recess or a recess above the end face 312 of the closure element 31, which is circumferentially bounded by the projection 34. By pressing the annular projection 34 into the crimping groove 14 when the cap element 10 is fitted, a press fit can be established on both sides and a particularly stable and reliable hold of the cap element 10 on the closure element 31 can be formed. In relation to the longitudinal axis L, the two contact surfaces 16, 17 are preferably closed in themselves in the shape of a ring or cylindrical shell, so that the crimping groove 14 is circumferential.

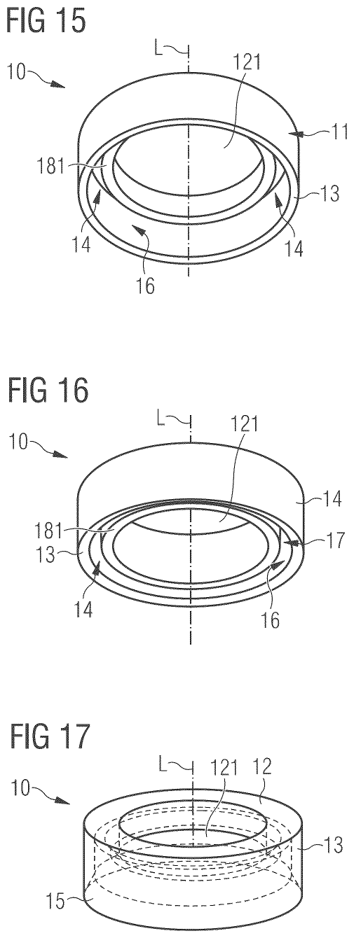

Furthermore, the first and/or the second contact surface 16, 17 can be formed with a predetermined inclination in relation to the longitudinal axis L and, in particular, run towards the other contact surface 16, 17 in the direction towards the end face 12 of the cap element 10. Inclined flanks, which can be realized by inclined contact surfaces 16, 17, enable easier placement on the projection 34 of the closure element 31. In FIG. 9, the first contact surface 16 is formed with an inclination, while the second contact surface 17 runs essentially parallel to the longitudinal axis L. In addition, the inner projection 18 has a rounding 181 adjacent to the second contact surface 17, which, analogous to the guide surface 15, enables simplified placement of the cap element 10 on the projection 34 of the closure element 31. Furthermore, the inner projection 18 can be matched with respect to a depth along the longitudinal axis L to the recess above the end face 312 of the closure element 31, which is bounded circumferentially by the projection 34. The inner projection 18 can be continuous, solid or annular (see also FIGS. 15 and 16).

FIG. 11 shows a transparent representation of a further embodiment of the cap element 10, which can be used on both sides. The cap element 10 has a first contact surface 16 and a second contact surface 17 on both the upper side 131 and the lower side 132 of the wall 13, each of which forms a pinch groove 14. The designs are separated from one another by the end wall 12, which according to this embodiment example establishes a center wall, but is arranged on top of the closure element 31 above the end wall 312 of the closure element 31. Accordingly, the cap element 10 can be coupled to both the upper side and the lower side by means of a press fit with the projection 34 of the closure element 31. The fact that the cap element 10 can be used on both sides enables a simplified application process, as it is no longer necessary to distinguish between the top and bottom when aligning the cap element 10. The cap element 10 can be designed inversely and thus have the same geometry of the contact surfaces 16, 17 at the top and bottom. Alternatively, as shown in FIG. 11, the cap element 10 can have a different geometry at the top than at the bottom. In the design at the top, the first contact surface 16 is inclined and runs in the direction of the end wall 12 towards the second contact surface 17, which is aligned essentially parallel to the longitudinal axis L. On the underside, both contact surfaces 16 and 17 are aligned essentially parallel to the longitudinal axis L and oriented towards each other.

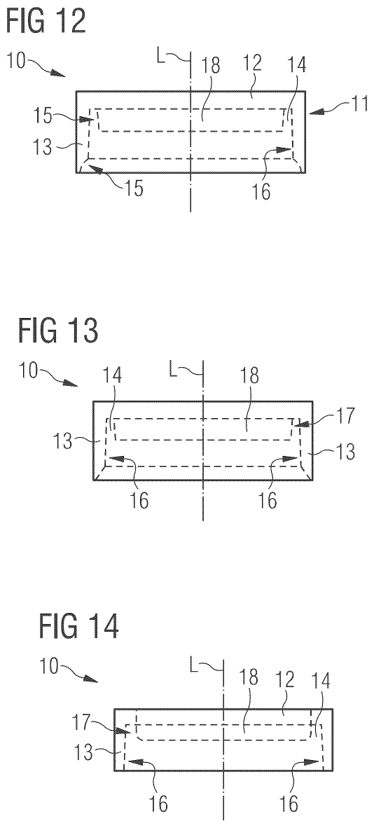

FIGS. 12 to 17 show further embodiments and/or views of the cap element 10. FIG. 12, for example, shows a sectional view of the cap element 10 with contact surfaces 16 and 17 converging towards each other, so that the pinch groove 14 becomes narrower towards the top. FIG. 13 illustrates the cap element 10 according to FIG. 12 in a transparent version. FIG. 15 shows the cap element 10 according to FIG. 7 in a perspective view from below. Compared to the embodiment according to FIG. 15, the cap element 10 according to FIG. 16 has an extended inner flank or second contact surface 17. In this way, a particularly secure fixation can be established by a relatively long pinch on both sides of the projection 34 on the closure element 31. FIG. 17 shows, for example, the cap element 10 according to FIGS. 7 and 15 in a transparent representation and a perspective view from above.

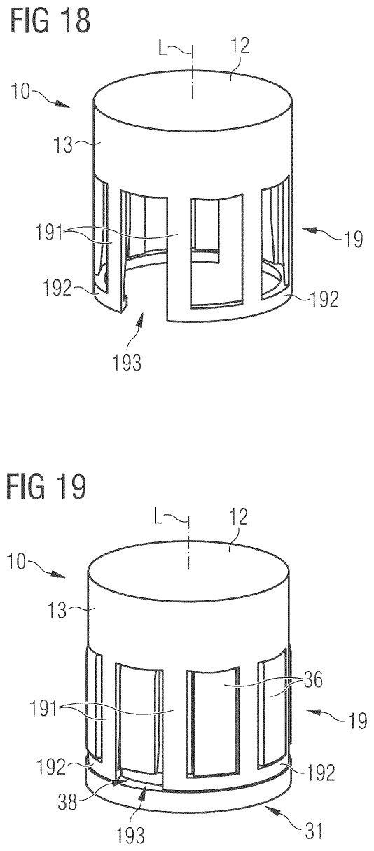

FIGS. 18 to 22 show further possible embodiments of the cap element 10 with a web structure 19, which is formed in particular in coordination with the existing structural features of the closure element 31. The web structure 19 comprises a plurality of guide webs 191, which are arranged to engage in the spaces 37 between the handle bars 36 of the closure element 31. The guide ridges 191 thus guide the seating of the cap element 10 when pressed onto the closure element 31 and can also contribute to a flush or balanced circumference on the closure element 31, which does not protrude outwardly and provides an enlarged adhesive surface that can be reliably labeled (see FIGS. 19 and 21). The engaging guide bars 191 also provide an anti-rotation lock around the longitudinal axis.

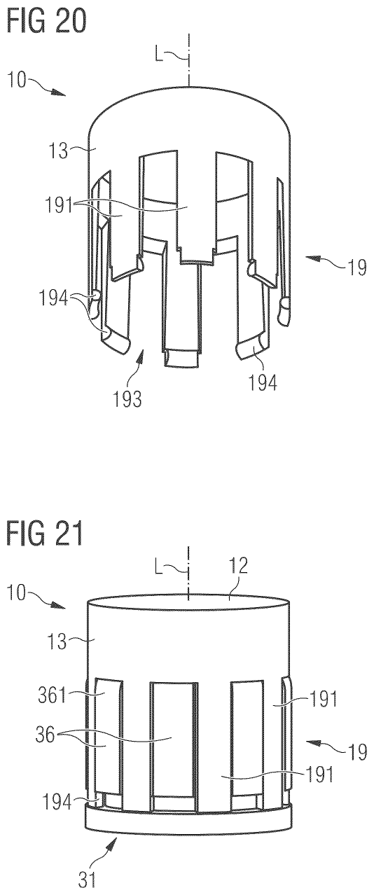

The web structure 19 according to FIGS. 18 and 19 has two latching webs 192, which form a respective latching element on the underside of the cap element 10. The latching webs 192 connect a plurality of guide webs 191 to one another, in the present embodiment example four guide webs 191 in each case. Two free spaces or openings 193 are provided between the latching webs 192, so that there is no circumferential, self-contained latching web. Thus, a useful elasticity of the web structure 19 can be set up so that the cap element 10 can be guided over the grip webs 36 of the closure element 31. In alternative embodiments, there may also be only one opening 193 or three or more openings 193, each of which provides a range of outward movement of the web structure 19 (see also FIGS. 20-22). The guide webs 191 connected by means of the locking webs 192 have a higher stability than guide webs 191 which are not connected at their lower ends. The latching webs 192 have an inwardly raised latching edge or latching projection 194 (see also FIGS. 20 and 21), which is set up to engage in the groove 38 of the closure element 31, so that a retaining force can be set up in the direction of the longitudinal axis L between the cap element 10 and the closure element 31 with respect to the longitudinal axis L. The engagement edge thus protrudes inwards compared to the adjacent sections of the guide webs 191.

FIGS. 20 and 21 show a further embodiment of the web structure 19 with a plurality of guide webs 191, at the lower end of which in each case a latching element is formed in the form of a latching edge or a latching projection 194. The respective latching projection 194 protrudes inwards and is set up to engage in the groove 38 of the closure element 31, so that a retaining force can be set up in the direction of the longitudinal axis L between the cap element 10 and the closure element 31 with respect to the longitudinal axis L.

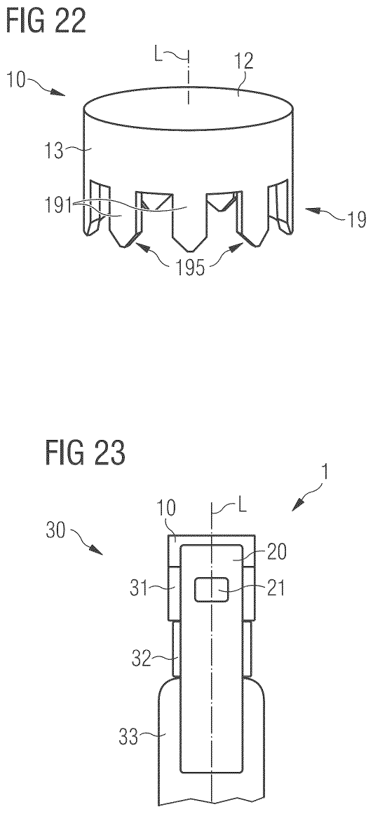

FIG. 22 shows a further embodiment example of the web structure 19 with a plurality of guide webs 191, which have a shortened design compared to the longer guide webs 191 according to FIG. 20 and each have guide surfaces 195 at their lower, free ends, which contribute to a simplified insertion of the guide webs 191 into the spaces 37 and placement of the cap element 10 on the closure element 31. According to this embodiment example, the beveled guide webs 191 have an arrow or cone shape.

FIGS. 23 to 25 show another embodiment example of the system 1 with the labeling arrangement comprising the cap element 10 and the label 20, which are attached to the syringe 30. The label 20 has an RFID functional unit and can accordingly also be referred to as an RFID label 20. The labeling arrangement preferably comprises an adhesive layer arranged on an underside of the RFID label 20. The RFID functional unit enables electronic storage and/or reading of information by means of the RFID chip 21 and the antenna structure. The RFID functional unit can, for example, be adhered to or on the cap element 10 by applying a hard tag or soft tag, for example as part of the RFID label 20. Alternatively, an RFID functional unit can already be integrated into the cap element 10 during its manufacture, for example by means of molding or so-called “in-mold” technologies.

The RFID label 20 is arranged on the syringe 30 in such a way that it is attached to the pressed-on cap element 10 on the one hand and to the syringe body 33 on the other. As a wrap-around or wrap-around label, the RFID label 20 can close or seal the syringe 30 at the top and bottom and counteract the penetration of unwanted particles or unauthorized manipulation. An intermediate area, such as the threaded element 32, is bridged and, for example, partially bonded.

In particular, the cap element 10 is designed and geometrically matched to the closure element 31 in such a way that it does not lead to a distortion of the syringe geometry. The widest point of the illustrated Luer-Lock syringe 30 according to FIGS. 1, 24 and 25 is given, for example, by the handle bars 36 or a collar below the groove 38 in the transition to the Luer adapter 32. The cap element 10 is therefore adapted to the narrower point at the head end of the closure element 31, which has the projection 34.

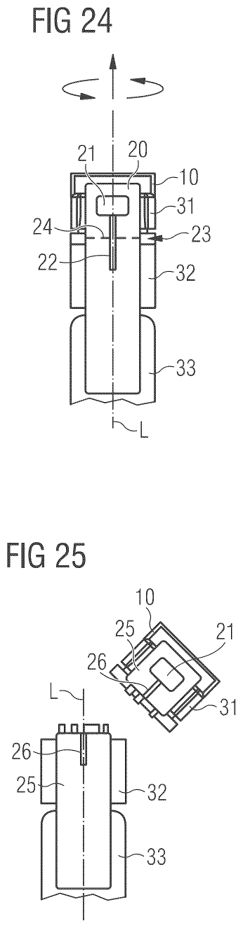

The label 20 can also have or provide an opening detection element that is arranged in a separation area 23 of the label 20. The opening detection element comprises, for example, a perforation 24 which, as a severing element, enables a controlled and predetermined severing of the label 20 in the severing area 23. Thus, by removing a partial element of the label 20 from the syringe 30, an opening of the syringe applied with the labeling arrangement can be visually indicated. The removable partial element can be formed, for example, as a label section 25 and/or as a conductor track section 26 of a conductor track 22 of the RFID functional unit (see FIGS. 24 and 25). In the area of the perforation 24, the label 20 can, for example, be designed to be non-sticky in order to facilitate the separation of the label 20 and removal of a label section 25 and any conductor track 22 present when opening the syringe 30.

The conductor track 22 alternatively or additionally provides an electronic opening detection element in form. The conductor track 22 is preferably placed over a cut line of the RFID label 20 and is formed, for example, by printing a conductive paste or as a component of the antenna structure of the RFID functional unit. The conductor track 22 can be connected to the RFID chip 21 of the RFID functional unit. When the syringe 30 is opened, the conductor track 22 is destroyed (see FIG. 25) and conductor track sections 26 remain on the separated elements. Such a destruction of the specifically arranged conductor track 22 can be registered by the RFID chip 21 as a detuning of the antenna structure or as a clear change in the reading range. Thus, as an alternative or in addition to mechanical versions of an opening indicator, an electronic first-opening indicator and tamper protection can be realized by means of the labeling arrangement.

The cap element 10 can also be designed in such a way that it specifically increases the length of the syringe 30 (see FIG. 10). This makes it possible, for example, for the RFID functional unit attached to the cap element 10 to be arranged closer to a bottom of a syringe barrel, in which a plurality of syringes 30 can be accommodated upside down, and can thus be detected more reliably by readers that are intended to read the RFID chips 21 from a bottom side of the syringe barrel.

The cap element 10 can also be designed with a predetermined surface structure on the outer surface 11, which is optimized or adapted for the application of the label 20. This can be achieved, for example, by a sufficiently large and suitably shaped flat surface of the cap element 10. The RFID functional unit can be applied as an RFID label 20 by means of pressure-sensitive adhesive to such a predetermined surface, whereby it is advantageous if a material of the cap element 10 has a surface tension suitable for the adhesion of a pressure-sensitive adhesive and/or the outer surface 11 has a microstructure suitable for the adhesion of a pressure-sensitive adhesive.

A method for applying or attaching an embodiment of the labeling arrangement or for forming the system 1 can be carried out according to the flowchart in FIG. 26 as follows:
In a step S1, the required components of the labeling arrangement and the elements of the syringe 30 to be coupled thereto are provided with predetermined and/or coordinated properties.

In a step S2, the cap element 10 is coupled to the closure element 31 by means of pressing on, so that the first contact surface 16 contacts and surrounds the projection 34 on the outside. If the cap element 10 has the second contact surface 17 and the resulting crimping groove 14, the cap element 10 is pressed onto the annular projection 34 until it is pressed into the crimping groove 14 and is connected to the cap element 10 by means of a press fit on both sides.

According to step S1, the closure element 31 can be provided individually or already arranged on the syringe 30. If the closure element 31 has been provided individually, the syringe body 33 of the syringe 30 can also be provided in a further step S3 and coupled to the closure element 31. The syringe body 33 may already be coupled to the threaded element 32 or may also be coupled to it in this step.

In a further step S4, the label 20, which for example has an RFID functional unit, is attached. In particular, the RFID label 20 is coupled or applied adhesively, so that the RFID label 20 is adhered to the top of the cap element 10 and to the bottom of the syringe body 33.

By means of the described labeling arrangement and the special cap element 10, structural features on the top of the syringe cap or the closure element 31 are specifically used for mechanical attachment. This makes it possible for the cap element 10 as an adapter cap to cover only the uppermost area of the syringe cap and to be firmly interlocked or pressed with it. In commercially available syringes, the topmost area is generally thinner in diameter than the syringe body 33. It is a finding of the present invention that this narrow area for the cap element 10 can be used to produce at least approximate circumferential equality with the syringe body 33 at a head end on a thin syringe 30. Accordingly, the cap element 10 can be manufactured with a wall thickness sufficient for manufacturability and mechanical stability and still establish circumferential uniformity in the applied area with the syringe body 33. The cap element 10 preferably provides sufficiently smooth surfaces for a particularly reliable adhesion of the label 20, so that the label 20, which connects the cap element 10 to the syringe body 33, can be applied sufficiently firmly.

By means of the pressed-on cap element 10, a sufficiently firm coupling for force transmission in the rotational and axial direction can be implemented with the syringe cap or the closure element 31, so that the syringe cap can be removed together with the cap element 10 when the syringe 30 is opened. In particular, the designs of the cap element 10 take into account structural features that are independent of the rotational position, such as the annular protrusion 34 on the upper side of the syringe cap. The corresponding structural features of the cap element 10, such as the first contact surface 16 and/or the pinch groove 14, are also independent of the rotational position in this respect. This means that the cap element 10 can also be applied independently of the rotational position, for example by simply and stably pressing it on from above without aligning the syringe 30 or the closure element 31, so that an application process is significantly simplified. In particular, a wedge-shaped crimping groove 14 facilitates the application process due to its self-adjusting design and leads to a particularly firm and stable wedging.

### LIST OF REFERENCE SIGNS

1 system
10 cap element
11 outer side/outer wall of the cap element
12 end face/end wall of the cap element
121 opening of the end wall
13 wall of the cap element
131 top side of the wall
132 underside of the wall
14 pinch groove of the cap element
15 guide surface/contact surface of the cap element
16 first contact surface of the cap element
17 second contact surface of the cap element
18 inner projection of the cap element
181 rounding of the inner projection
19 web structure of the cap element
191 guide bar of the bar structure
192 locking bar of the bar structure
193 opening of the bar structure
194 latching projection of the web structure
195 guide surface of the guide bar
20 label/RFID label
21 RFID chip
22 electrical conductor track
23 separation area
24 perforation
25 label section
26 conductor section
30 container/syringe
31 closure element of the container/syringe cap of the syringe
311 outer side/outer wall of the closure element
312 end face/end wall of the closure element
32 threaded element of the container/Luer adapter of the syringe
33 container body/syringe body
34 annular projection of the closure element
35 syringe plunger
36 handle bar of the closure element
361 bar taper
37 intermediate space
38 groove of the closing element
L longitudinal axis of the syringe/cap element
S(i) step of a method for attaching a cap element/labeling arrangement

1. A labeling arrangement for a multi-part container (30), comprising:
a sleeve-shaped cap element (10) which comprises a wall (13) with an inner surface which establishes a first contact surface (16) and which is designed to be coupled, relative to a longitudinal axis (L) of the container (30), by means of an interference fit to an outer surface (311, 312) of a closure element (31) of the container (30) and to surround the closure element (31).

2. The labeling arrangement according to claim 1, wherein the cap element (10) comprises an inner projection (18) which establishes a second contact surface (17), the first and second contact surfaces (16, 17) being formed in coordination with each other and in coordination with an annular projection (34) on an end face (312) of the closure element (31) and providing a coupling structure in the form of a press groove (14) which can be coupled to the annular projection (34) by means of a press fit.

3. The labeling arrangement according to claim 2, wherein the first and/or the second contact surface (16, 17) is designed to be annularly closed in itself with respect to the longitudinal axis (L) and sets up the press groove (14) in a circumferential manner.

4. The labeling arrangement according to claim 2, wherein the first and/or the second contact surface (16, 17) is designed with a predetermined inclination with respect to the longitudinal axis (L) and runs towards the first or second contact surface (16, 17) in the direction of an end face (12) of the cap element (10).

5. The labeling arrangement according to claim 1, wherein the cap element (10) has a penetrating opening (121) at an end face (12).

6. The labeling arrangement according to claim 1, wherein the cap element (10) has a guide surface (15) on an underside (132) of the wall (13) adjacent to the first contact surface (16) for placing on the closure element (31).

7. The labeling arrangement according to claim 6, wherein the guide surface (15) is further formed in coordination with an outer contour of the cap element (31) to establish a flush transition between the cap element (10) and the closure element (31).

8. The labeling arrangement according to claim 2, wherein the inner protrusion (18) has a rounding (181) on an underside adjacent to the second contact surface (17) for placement on the closure element (31).

9. The labeling arrangement according to claim 1, wherein the cap element (10) has a first contact surface (16) both on an upper side (131) and on an underside (132) of the wall (13), which are separated from each other by an end wall, so that the cap element (10) can be coupled by means of a press fit to the outer surface (311, 312) of the closure element (31) both with the upper side (131) and with the lower side (132) with respect to the longitudinal axis (L) of the container (30).

10. The labeling arrangement according to claim 1, wherein the cap element (10) comprises a web structure (19) which is formed in coordination with an outer contour of the closure element (31) of the container (30), wherein the web structure (19) comprises a plurality of webs (191) which are adapted to engage in corresponding spaces (37) between grip webs (36) of the closure element (31).

11. The labeling arrangement according to claim 10, wherein one or more webs (191) of the web structure (19) have a latching element in the form of a latching web (192), which connects at least two webs (192) to one another, and/or in the form of a latching projection (194), which is formed at a free end of a web (191), wherein the respective latching element is set up to engage in a groove (38) of the closure element (31) of the container (30), so that a retaining force can be set up in the direction of the longitudinal axis (L) between the cap element (10) and the closure element (31) relative to the longitudinal axis (L).

12. The labeling arrangement according to claim 1, wherein the cap element (10) has a circumferential contour with respect to the longitudinal axis (L), which contour forms a negative shape with respect to a circumferential contour of the closure element (31).

13. The labeling arrangement according to claim 1, wherein the cap element (10) is designed such that it extends only in the region of the closure element (31).

14. The labeling arrangement according to claim 1, wherein the wall (13) of the cap element (10) has a thickness of 0.5 to 3.0 millimeters with respect to a radial extension transverse to the longitudinal axis (L).

15. The labeling arrangement according to claim 1, comprising:
a label (20), wherein the label (20) and the cap element (10) are formed in coordination with each other and can be coupled to each other, so that the label (20) can be attached to the closure element (31) of the container (30) by means of the cap element (10).

16. The labeling arrangement according to claim 15, wherein the label (20) comprises an RFID functional unit with an RFID chip (21) and an antenna structure coupled thereto.

17. The labeling arrangement according to claim 15, comprising:
an opening detection element which is arranged in a separation region (23) of the label (20) and is designed to indicate an opening of the container (30) applied with the labeling arrangement by means of removing at least one partial element (25, 26) of the label (20) from the container (30), wherein the at least one removable partial element is designed as a label section (25) and/or a conductor track section (26) of the RFID functional unit.

18. A system (1), comprising:
a container (30) which can be divided into a plurality of parts and has a closure element (31) and a container body (33) which are coupled to one another, and
the labeling arrangement according to claim 1, wherein the cap element (10) is coupled to the closure element (31) by means of an interference fit, so that the first contact surface (16) contacts an outer surface (311, 312) of the closure element (31) and surrounds the closure element (31).

19. The system (1) according to claim 18, wherein the labeling arrangement comprises a label (20) attached to the cap element (10) and to the container body (33).

20. A method for attaching a labeling arrangement according to claim 1 to a closure element (31) of a multi-part container (30), comprising:
providing the container (30) with the closure element (31) and a container body (33) coupled to each other,
providing the sleeve-shaped cap element (10),
placing and pressing the cap element (10) onto the closure element (31) so that the first contact surface (16) contacts an outer surface (311, 312) of the closure element (31) and the cap element (10) is attached to and surrounds the closure element (31) by means of an interference fit.
